(19) United States
(12) Patent Application Publication
(10) Pub. No.: US 2020/0305964 A1
(43) Pub. Date: Oct. 1, 2020

(54) BLADE ESCAPE PREVENTION FEATURE FOR SURGICAL INSTRUMENT AND METHOD FOR PREVENTING SAME

(71) Applicant: Covidien LP, Mansfield, MA (US)

(72) Inventors: David D. Brause, Longmont, CO (US); Monte S. Fry, Longmont, CO (US); Curtis M. Siebenaller, Frederick, CO (US)

(21) Appl. No.: 16/833,023

(22) Filed: Mar. 27, 2020

Related U.S. Application Data
(60) Provisional application No. 62/825,887, filed on Mar. 29, 2019.

(51) Int. Cl. A61B 18/14

(52) U.S. Cl. CPC A61B 18/1445; A61B 2017/00867

(57) ABSTRACT

A jaw member for a surgical instrument includes an outer housing having a sealing plate operably coupled thereon including an upper knife channel defined therethrough and extending therealong. A pair of opposing jaw flanges is configured to operably couple to a corresponding pair of jaw flanges of an opposing jaw member. An insulative insert is disposed within the outer housing and includes opposing sidewalls and a connector portion which define an engagement channel and a lower knife channel in vertical registration with the upper knife channel, the upper and lower knife channels configured to receive a knife of a knife assembly, the engagement channel configured to receive a tube of the knife assembly. A top plate is engaged between the jaw flanges, a portion of the top plate is configured to extend across the engagement channel and retain the tube within the engagement channel during translation of the knife assembly.

### CROSS REFERENCE TO RELATED APPLICATIONS

This application claims the benefit of and priority to U.S. Provisional Patent Application No. 62/825,887, filed on Mar. 29, 2019, the entire content of which is incorporated by reference herein.

### BACKGROUND

### Technical Field

The present disclosure relates to surgical instruments, and more particularly, to a blade escape prevention feature and method for preventing blade escape for use with an articulating surgical forceps.

### Background of Related Art

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife or cutting member utilized to effectively sever the treated tissue.

Many electrosurgical forceps include various actuators to orient the jaw members for tissue treatment. For example, many forceps include rotational wheels (or the like) disposed in proximity to a surgeon's hands to enable the surgeon to selectively rotate the jaw members as needed during an operation. A trigger (or similar) may be disposed on the forceps housing to allow a surgeon to selectively deploy a knife or cutting element as needed during surgery. Other actuators include articulating mechanisms disposed in proximity to the surgeon's hands to allow the surgeon to selectively articulate (e.g., pitch and yaw) the jaw members as needed during surgery.

With particular respect to articulating forceps that include a deployable knife, one important feature of these types of forceps is the knife drive rod or drive member which typically needs to be both sufficiently flexible to allow articulation of the jaw members while also being strong enough to advance and retract a knife blade through tissue. In some instances, since the knife needs to be sufficiently strong enough to cut various tissue types and the knife drive rod needs to be sufficiently flexible to both translate through the articulation joint and drive the knife, dissimilar metals may need to be used for the knife drive rod and the knife. Typically, the knife drive rod is welded to the knife. However, in certain instances, the knife drive rod to knife weld may need to be supplemented with additional engagement features to insure proper translation of the knife and minimize the risk of blade escape if the weld fails.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

In accordance with aspects of the present disclosure, a jaw member for a surgical instrument includes an outer housing having a tissue sealing plate operably coupled thereon, the tissue sealing plate including an upper knife channel defined therethrough and extending therealong. A pair of opposing jaw flanges is disposed proximally of the tissue sealing plate and is configured to operably couple to a corresponding pair of jaw flanges of an opposing jaw member. An insulative insert is disposed within the outer housing and includes first and second opposing sidewalls and a connector portion which define an engagement channel and a lower knife channel in vertical registration with the upper knife channel. The upper and lower knife channels are configured to slidingly receive a knife of a knife assembly and the engagement channel is configured to slidingly receive a tube of the knife assembly or similar mechanical retention feature. A top plate is engaged to the outer housing between the jaw flanges. A portion of the top plate is configured to extend across the engagement channel in opposition to the connection portion and retain the tube within the engagement channel during translation of the knife assembly.

In aspects according to the present disclosure, the upper and lower knife channels and the engagement channel are curved along a vertical axis defined through the jaw member to prevent trapping tissue. In other aspects according to the present disclosure, the length of the knife channel is longer than the length of the engagement channel. In still other aspects according to the present disclosure, the top plate is welded between the jaw flanges.

In aspects according to the present disclosure, the knife of the knife assembly is made from a superelastic material. In other aspects according to the present disclosure, the superelastic material is Nitinol, Stainless Steel or High Carbon Steel Inconel, Monel, Nimonic, Nitronic, Hastelloy (Nickel based alloys other than NiTiNOL), Elgiloy (Cobalt-Nickel), Brass, Phosphor Bronze, Beryllium Copper, Chrome-Vanadium or Chrome-Silicon, Titanium, Braided Cable (i.e. Steel or Tungston).

In aspects according to the present disclosure, the knife assembly includes a knife drive rod or drive member, the knife and the tube. In other aspects according to the present disclosure, the tube is welded (or crimped, bonded or swaged) to a distal end of the knife drive rod to create a first weld joint. In yet other aspects according to the present disclosure, the tube of the knife assembly is welded within a cavity defined within the knife to create a second weld joint. In still other aspects according to the present disclosure, the first weld joint is configured to fail under stress prior to the second weld joint.

In accordance with aspects of the present disclosure, a jaw assembly for a surgical instrument includes first and second jaw members disposed in opposing relation thereto, one or both of the first or second jaw members includes an outer housing having a tissue sealing plate operably coupled thereon. A knife is included having a cavity defined therein. A knife drive rod includes a tube welded to a distal end thereof to form a first weld joint, the tube is secured within the cavity by a second weld joint. The first weld joint is configured to fail under stress prior to the second weld joint.

In aspects according to the present disclosure, the tissue sealing plate of the one or both jaw members includes an upper knife channel defined therethrough and extending therealong. In other aspects according to the present disclosure, the knife assembly further includes an insulative insert disposed within the outer housing having first and second opposing sidewalls and a connector portion which define an engagement channel and a lower knife channel in vertical registration with the upper knife channel. The upper and lower knife channels are configured to slidingly receive the knife of the knife assembly and the engagement channel is configured to slidingly receive the tube of the knife assembly.

In aspects according to the present disclosure, a top plate is engaged to the outer housing. A portion of the top plate is configured to extend across the engagement channel in opposition to the connection portion and retain the tube within the engagement channel during translation of the knife assembly. In other aspects according to the present disclosure, one or both of the first or second jaw members includes a pair of jaw flanges at a proximal end thereof and the top plate is welded between the jaw flanges of the one or both jaw members.

In aspects according to the present disclosure, the knife of the knife assembly is made from a superelastic material. In other aspects according to the present disclosure, the superelastic material is Nitinol [Stainless Steel or High Carbon Steel Inconel, Monel, Nimonic, Nitronic, Hastelloy (Nickel based alloys other than NiTiNOL), Elgiloy (Cobalt-Nickel), Brass, Phosphor Bronze, Beryllium Copper, Chrome-Vanadium or Chrome-Silicon, Titanium, Braided Cable (i.e. Steel or Tungston).

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements and:

FIG. 1A is a perspective view of endoscopic surgical forceps exemplifying the aspects and features of the present disclosure, wherein the shaft of the endoscopic surgical forceps is disposed in a non-articulated position and wherein the jaw members of the endoscopic surgical forceps are disposed in a spaced-apart position;

FIG. 1B is a perspective view of the endoscopic surgical forceps of FIG. 1A, wherein the shaft of the endoscopic surgical forceps is disposed in an articulated position and wherein the jaw members of the endoscopic surgical forceps are disposed in an approximated position;

FIGS. 2A, 2B and 2C are enlarged schematic views of one embodiment of an engagement feature for coupling a knife blade to a knife drive rod or member exemplifying the aspects and features of the present disclosure; and

FIG. 3A is a rear perspective view of one of the jaw members for use with the endoscopic surgical forceps of FIG. 1A;

FIG. 3B is an enlarged, rear perspective cross section of the jaw member of FIG. 3A taken along line 3B-3B; and

FIG. 4 is an enlarged schematic view of another embodiment of an engagement feature for coupling a knife blade to a knife drive rod exemplifying the aspects and features of the present disclosure.

### DETAILED DESCRIPTION

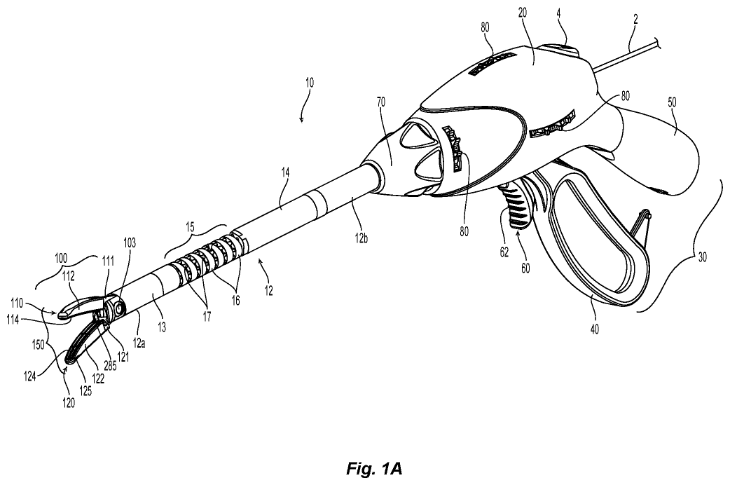

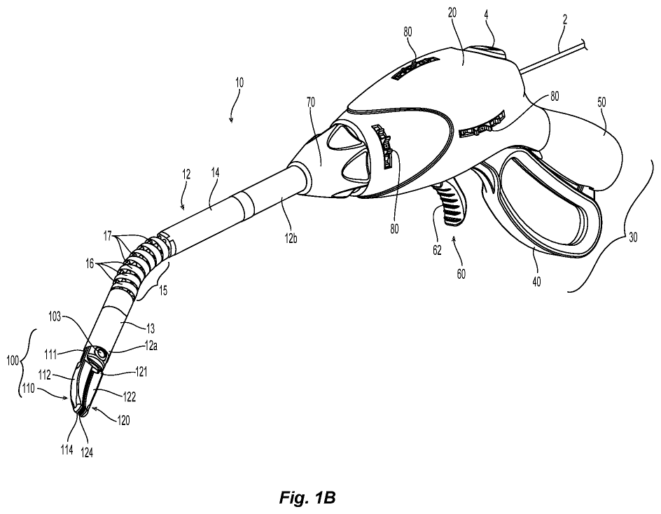

Referring generally to FIGS. 1A and 1B, an endoscopic surgical forceps exemplifying the aspects and features of the present disclosure is shown generally identified by reference numeral 10. For the purposes herein, endoscopic surgical forceps 10 is generally described. Aspects and features of endoscopic surgical forceps 10 not germane to the understanding of the present disclosure are omitted to avoid obscuring the aspects and features of the present disclosure in unnecessary detail.

Forceps 10 includes a housing 20, a handle assembly 30, a trigger assembly 60, a rotating assembly 70, a plurality of articulation actuators 80, an activation switch 4, and an end effector assembly 100. Forceps 10 further includes a shaft 12 having a distal end 12a configured to mechanically engage end effector assembly 100 and a proximal end 12b that mechanically engages housing 20. Forceps 10 also includes cable 2 that connects forceps 10 to an energy source (not shown), e.g., a generator or other suitable power source, although forceps 10 may alternatively be configured as a battery-powered device. Cable 2 includes a wire (or wires) (not shown) extending therethrough that has sufficient length to extend through shaft 12 in order to provide energy to one or both tissue-treating plates 114, 124 of jaw members 110, 120, respectively, of end effector assembly 100. Activation switch 4 is coupled to tissue-treating plates 114, 124 and the source of energy for selectively activating the supply of energy to jaw members 110, 120 for treating, e.g., cauterizing, coagulating/desiccating, and/or sealing, tissue.

Shaft 12 of forceps 10 defines a distal segment 13 positioned towards distal end 12a thereof, a proximal segment 14 positioned towards proximal end 12b thereof, and an articulating section 15 disposed between the distal and proximal segments 13, 14, respectively. Articulating section 15 includes a plurality of articulating links 16 having a plurality of articulation cables 17 extending therethrough. Each cable 17 is operably engaged at a distal end thereof to distal segment 13 and at a proximal end thereof to one of the articulation actuators 80 to enable articulation of distal segment 13 and, thus, end effector assembly 100, relative to proximal segment 14 upon actuation of one or more of articulation actuators 80. Rotating assembly 70 operably couples shaft 12 to housing 20 to enable selective rotation of shaft 12 and, thus, end effector assembly 100, relative to housing 20.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position (FIG. 1A) and an approximated position (FIG. 1B) to grasp tissue between jaw members 110, 120. As shown in FIG. 1A, movable handle 40 is initially spaced-apart from fixed handle 50 and, correspondingly, jaw members 110, 120 of end effector assembly 100 are disposed in the spaced-apart position. Movable handle 40 is compressible from this initial position to a compressed position corresponding to the approximated position of jaw members 110, 120 (FIG. 1B).

Trigger assembly 60 includes a trigger 62 coupled to housing 20 and movable relative thereto between an un-actuated position and an actuated position. Trigger 62 is operably coupled to a cutting mechanism 85, various embodiments of which are detailed below, to actuate the cutting mechanism 85 to cut tissue grasped between jaw members 110, 120 of end effector assembly 100 upon actuation of trigger 62. As an alternative to a pivoting trigger 62, a slide trigger, push-button, toggle switch, or other suitable actuator may be provided.

End effector assembly 100, as noted above, includes first and second jaw members 110, 120. Each jaw member 110, 120 includes a proximal flange portion 111, 121, an outer insulative jaw housing 112, 122 disposed about the distal portion (not explicitly shown) of each jaw member 110, 120, and a tissue-treating plate 114, 124, respectively. Proximal flange portions 111, 121 are pivotably coupled to one another about pivot 103 for moving jaw members 110, 120 between the spaced-apart and approximated positions, although other suitable mechanisms for pivoting jaw members 110, 120 relative to one another are also contemplated. The distal portions (not explicitly shown) of the jaw members 110, 120 are configured to support jaw housings 112, 122, and tissue-treating plates 114, 124, respectively, thereon.

Outer insulative jaw housings 112, 122 of jaw members 110, 120 support and retain tissue-treating plates 114, 124 on respective jaw members 110, 120 in opposed relation relative to one another. Tissue-treating plates 114, 124 are formed from an electrically conductive material, e.g., for conducting electrical energy therebetween for treating tissue, although tissue-treating plates 114, 124 may alternatively be configured to conduct any suitable energy, e.g., thermal, microwave, light, ultrasonic, etc., through tissue grasped therebetween for energy-based tissue treatment. As mentioned above, tissue-treating plates 114, 124 are coupled to activation switch 4 and the source of energy (not shown), e.g., via the wires (not shown) extending from cable 2 through forceps 10, such that energy may be selectively supplied to tissue-treating plate 114 and/or tissue-treating plate 124 and conducted therebetween and through tissue disposed between jaw members 110, 120 to treat tissue. An overmold 139 is configured to capture the tissue-treating plate 124 during assembly.

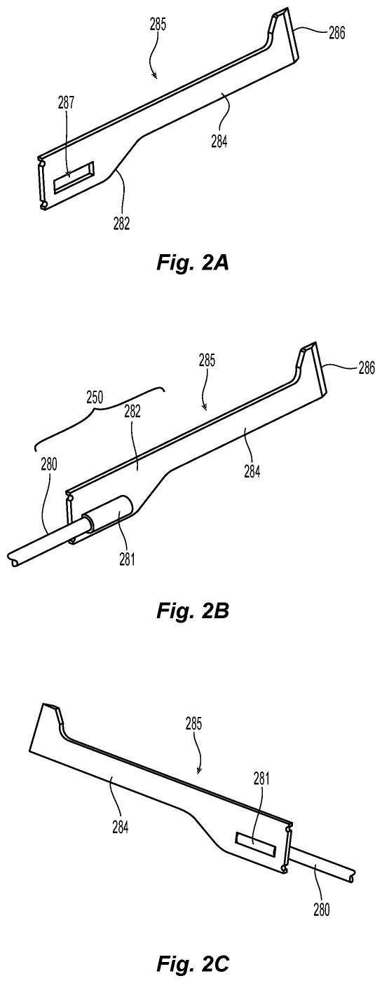

One or both of jaw members 110, 120 may further define a longitudinally-extending knife channel 125 (only the channel 125 of jaw member 120 is shown) for allowing reciprocation of the cutting mechanism 85 upon actuation of trigger 62. Actuation of the trigger 62 reciprocates a knife drive bar or knife drive member, e.g., knife drive rod 280 of FIG. 2B, operably coupled to the cutting mechanism, e.g., knife 285 through knife channel 125. Knife drive rod 280 may include any type of rod or cable (braided or otherwise) utilized to deploy the knife, e.g., knife 285. Together and as used herein, the knife, e.g., knife 285 and knife drive rod, e.g., knife drive rod 280, form a knife assembly 250. Knife drive rod 280 is made from a flexible material of sufficient strength to allow the knife drive rod 280 to both push and pull the knife 285 through tissue disposed between jaw members 110, 120. Moreover, the flexibility of the knife drive rod 280 allows the knife drive rod 280 to flex as needed during articulation of the jaw members 110, 120. The knife drive rod 280 may be made from a variety of flexible materials such as Nitinol, Stainless Steel or High Carbon Steel Inconel, Monel, Nimonic, Nitronic, Hastelloy (Nickel based alloys other than NiTiNOL) that exhibit the necessary strength and flexibility to allow smooth translation of the knife drive rod 280 through one or more articulating joints of articulating section 15.

Knife 285 is typically made from a stronger material, e.g., stainless steel or High Carbon Steel, to allow the knife 285 to maintain sharpness, resist breakage and easily translate through tissue on a repeated basis,. Other known materials are also contemplated.

Since it is often difficult to assure a consistent and strong weld between two dissimilar metals, i.e., utilizing a flexible first material, e.g., Nitinol, for the knife drive rod 280 with a second stronger material for the knife 285, e.g., stainless steel, various welding and mechanical capture techniques are described below with respect to FIGS. 2A-4.

FIGS. 2A-2C show one embodiment of a knife 285 for engagement to a knife drive rod 280. More particularly, knife 285 includes a knife body 284 having a distal end 286 and a proximal end 282, the distal end 286 including a sharpened edge for cutting tissue and the proximal end 282 including a slot 287 defined therein and configured to capture a tube 281 crimped, threaded or welded onto a portion of the knife drive rod 280. Together the knife 285 and knife drive rod 280 (with the tube 281 attached thereto) form the knife assembly 250.

Since the knife drive rod 280 needs to be flexible to accommodate articulation of the jaw members 110, 120, and the knife body 284 needs to be sufficiently strong to cut through tissue on a repeated basis, the knife drive rod 280 and the knife body 284 are typically made from dissimilar materials and any such weld or bond may be weaker than desired. Thus additional mechanical engagement between the two elements, e.g., the knife drive rod 280 and knife body 284, is needed to prevent mechanical failure. Tube 281, on the other hand, may be made from any type of metal, e.g., stainless steel, that will provide a secure weld to knife body 284.

In embodiments, the knife body 284 and the tube 281 are made from the same material, e.g., stainless steel, to assure a good weld. In addition and during assembly the tube 281 is seated within slot 287 to capture the tube 281 therein and provide additional mechanical engagement between the knife drive rod 280 and the knife body 284 (See FIG. 2C). As mentioned above, the knife drive rod 280 and the tube 281 are typically made from dissimilar metals, e.g., Nitinol and stainless steel, respectively, and, when welded, may produce a weaker weld. In the particular embodiment of FIGS. 2A-2C, if the weaker weld between the knife drive rod 280 and the tube 281 fails, the stronger bond between the knife body 284 and the tube 281 will remain intact thereby preventing the possibility of the knife 285 coming out of one or both jaw members, e.g., jaw member 120.

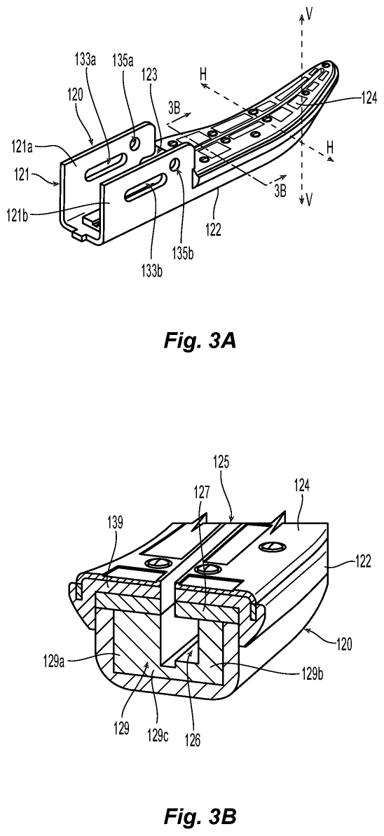

Turning now to FIGS. 3A and 3B (in conjunction with the embodiment shown in FIGS. 2A-2C), as mentioned above, jaw member 120 includes a proximal flange 121 that includes upwardly extending flanges or jaw flags 121a, 121b that define a cavity 123 therebetween configured to receive opposing proximal flange 111 (See FIG. 1A) of jaw member 110. Flanges 121a, 121b define respective cam slots 133a, 133b and pivot holes 135a, 135b for receiving a drive pin (not shown) and pivot 103. Jaw member 110 includes similar cam slots and pivot holes (both not shown).

As mentioned above, jaw member 120 defines a knife channel 125 defined therethrough that is dimensioned to allow reciprocation of knife 285 upon selective actuation thereof. Together the knife assembly 250 and the jaw members 110, 120 form a jaw assembly 150. The knife drive rod 280 and tube 281 are dimensioned to seat within a retention feature, e.g., an engagement channel 126, defined in the jaw member 120 adjacent the knife channel 125. Knife channel 125 is defined through both tissue sealing plate 124 (upper knife channel) and through an insulative insert 129 disposed underneath tissue sealing plate 124 (lower knife channel). Insulative insert 129 includes opposing side walls 129a, 129b and a bottom connector portion 129c which define both knife channel 125 and engagement channel 126. Engagement channel 126 is dimensioned to slidingly receive the tube 281 when engaged to the knife 285. Engagement channel 126 is configured to extend along the length of the knife channel 125 (or at least partially along the length of knife channel 125) and is configured to receive the tube 281 when engaged within the slot 287 of knife 285. As explained in more detail below, this provides both lateral and vertical stability to the knife 285 and safety enhancement.

More particularly, a top plate 127 covers the upper portion of engagement channel 126 and is configured to prevent the tube 281 (and the knife 285) from dislodging during translation thereof. This acts as a safety feature and alleviates concerns that the knife 285 will escape from the knife channel 125 during reciprocation especially when cutting dense tissue structures. Still further, in the event of drive rod 280 breaks or the weld joint between the tube 281 and the drive rod 280 fails, the tube 281 remains captured within the engagement channel 126 preventing the knife 285 from dislodging from the jaw member 120.

Moreover, the top plate 127 acts to resist the jaw member 120 and seal plate 124 from bending due to high tissue pressures. As shown best in FIG. 3B, the top plate 127 is welded to the jaw flanges 121a, 121b (and the main body of jaw member 120) to allow the top plate 127 to bear the brunt of the stress from tissue obstacles. Welding the top plate 127 to the jaw flanges 121a, 121b also prevents the jaw flanges 121a, 121b from bowing.

In addition, the strength of the tube 281 to knife 285 weld and the drive rod 280 to tube 281 engagement can be optimized to ensure that in the event the knife 285 encounters an obstacle putting excessive force on the knife 285, the engagement between the drive rod 280 and tube 281 will intentionally fail before the tube 281 to knife 285 weld (or the knife 285 breaks) essentially acting as a safety fuse to prevent parts of the knife 285 from coming free from the jaw members 120.

The engagement channel 126 may also be configured to limit the knife 285 travel within knife channel 125. More particularly, the engagement channel 126 may be dimensioned shorter than the knife channel 125 within the jaw member 120 and act as a stop to regulate distal movement of the knife 285. The knife channel 125 and the engagement channel 126 may be configured to curve relative to both a horizontal axis “H” and a vertical axis “V” through the jaw members, e.g., jaw member 120. A curved knife channel 125 and engagement channel 126 can be defined in either a straight jaw member 120 or a correspondingly curved jaw member 120. It is envisioned that curving the knife channel 125 and engagement channel 126 along the vertical axis “V” will prevent trapping tissue during multiple actuations.

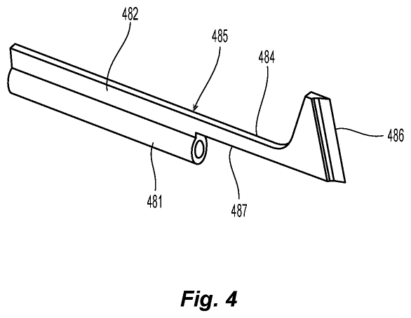

FIG. 4 shows another embodiment of a knife 485 for engagement to a knife drive rod (not shown). More particularly, knife 485 includes a knife body 484 having a distal end 486 and a proximal end 482, the distal end 486 including a sharpened edge for cutting tissue and the proximal end 482 configured to mechanically engage a tube 481 which may be crimped, threaded or welded (or formed as part of the blade itself) onto the proximal end 482 along a lower edge 487 of the knife body 484. Tube 481 may be made from any type of metal, e.g., stainless steel, that will provide a secure weld to knife body 484.

In embodiments, the knife body 484 and the tube 481 are made from the same material, e.g., stainless steel, to assure a good weld. The knife drive rod is secured within the tube 481 during assembly via crimping, welding or threadable engagement. Engaging the knife drive rod to the tube 481 which is secured to the lower edge 487 of the knife body 484 facilitates a more balanced actuation of the knife 485 during translation since the mechanical engagement of the knife body 484 and the tube 481 is along the centerline (lower edge 487) of the knife 485.

Jaw member 120 may include an engagement channel (not shown) similar to engagement channel 126 that is configured to accommodate the knife 485 and tube 481 embodiment shown in FIG. 4. More particularly, the lower portion of the insulative insert 129 may be dimensioned to define a knife channel and an engagement channel that mechanically interfaces with the knife 485 and tube 481 embodiment in FIG. 4 such that the tube 481 (located on the lower edge 487 of knife 485) is selectively translatable within the engagement channel. Again and as explained above, the mechanical engagement of the engagement channel and tube 481 provides additional strength to the knife 485 and knife body 484 and acts as a safety feature.

The various embodiments disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

For a detailed description of exemplary medical work stations and/or components thereof, reference may be made to U.S. Patent Application Publication No. 2012/0116416, and PCT Application Publication No. WO2016/025132, the entire contents of each of which are incorporated by reference herein.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A jaw member for a surgical instrument, comprising:
an outer housing including a tissue sealing plate operably coupled thereon, the tissue sealing plate including an upper knife channel defined therethrough and extending therealong;
a pair of opposing jaw flanges disposed proximally of the tissue sealing plate and configured to operably couple to a corresponding pair of jaw flanges of an opposing jaw member;
an insulative insert disposed within the outer housing and including first and second opposing sidewalls and a connector portion which define an engagement channel and a lower knife channel in vertical registration with the upper knife channel, the upper and lower knife channels configured to slidingly receive a knife of a knife assembly, the engagement channel configured to slidingly receive a tube of the knife assembly; and
a top plate engaged to the outer housing between the jaw flanges, a portion of the top plate configured to extend across the engagement channel in opposition to the connection portion and retain the tube within the engagement channel during translation of the knife assembly.

2. The jaw member for a surgical instrument according to claim 1, wherein the upper and lower knife channels and the engagement channel are curved along a vertical axis defined through the jaw member to prevent trapping tissue.

3. The jaw member for a surgical instrument according to claim 1, wherein the length of the knife channel is longer than the length of the engagement channel.

4. The jaw member for a surgical instrument according to claim 1, wherein the top plate is welded between the jaw flanges.

5. The jaw member for a surgical instrument according to claim 1, wherein the knife of the knife assembly is made from a superelastic material.

6. The jaw member for a surgical instrument according to claim 5, wherein the superelastic material is Nitinol.

7. The jaw member for a surgical instrument according to claim 1, wherein the knife assembly includes a knife drive rod, the knife and the tube.

8. The jaw member for a surgical instrument according to claim 7, wherein the tube is welded to a distal end of the knife drive rod to create a first weld joint.

9. The jaw member for a surgical instrument according to claim 8, wherein the tube of the knife assembly is welded within a cavity defined within the knife to create a second weld joint.

10. The jaw member for a surgical instrument according to claim 9, wherein the first weld joint is configured to fail under stress prior to the second weld joint.

11. A jaw assembly for a surgical instrument, comprising:
first and second jaw members disposed in opposing relation thereto, at least one of the first or second jaw members including an outer housing having a tissue sealing plate operably coupled thereon;
a knife including a cavity defined therein;
a knife drive rod including a tube welded to a distal end thereof to form a first weld joint, the tube secured within the cavity by a second weld joint, wherein the first weld joint is configured to fail under stress prior to the second weld joint.

12. The jaw assembly for a surgical instrument according to claim 11, wherein the tissue sealing plate of the at least one jaw member includes an upper knife channel defined therethrough and extending therealong.

13. The jaw assembly for a surgical instrument according to claim 12, further comprising: an insulative insert disposed within the outer housing having first and second opposing sidewalls and a connector portion which define an engagement channel and a lower knife channel in vertical registration with the upper knife channel, the upper and lower knife channels configured to slidingly receive the knife of the knife assembly, the engagement channel configured to slidingly receive the tube of the knife assembly.

14. The jaw assembly for a surgical instrument according to claim 13, further comprising:
a top plate engaged to the outer housing, a portion of the top plate configured to extend across the engagement channel in opposition to the connection portion and retain the tube within the engagement channel during translation of the knife assembly.

15. The jaw assembly for a surgical instrument according to claim 13, wherein at least one of the first or second jaw members includes a pair of jaw flanges at a proximal end thereof and wherein the top plate is welded between the jaw flanges of the at least one jaw member.

16. The jaw assembly for a surgical instrument according to claim 11, wherein the knife of the knife assembly is made from a superelastic material.

17. The jaw assembly for a surgical instrument according to claim 16, wherein the superelastic material is Nitinol.
